SEQUENCE LISTING (2 sequences)

SEQ ID NO: 1 - protein, 594 aa, Shewanella halifaxensis
MSRLLLCICVLLFSSVAWSKPQQFYVGVLANWGHQQAVERWTPMMEYLNEHVPDAEFHVYPGNFKALNLAMELGQIQFIITNPGQYLYLSNQYPLSWLATMRSKRHDGTTSAIGSAIIVRADSDYRTLYDLKGKVVAASDPHALGGYQATVGLMHSLGMDPDTFFGETKFLGFPLDPLLYQVRDGNVDAAITPLCTLEDMVARGVLKSSDFRVLNPSRPDGVECQCSTTLYPNWSFAATESVSTELSKEITQALLELPSDSPAAIKAQLTGWTSPISQLAVIKLFKELHVKTPDSSRWEAVKKWLEENRHWGILSVLVFIIATLYHLWIEYRFHQKSSSLIESERQLKQQAVALERLQSASIVGEIGAGLAHEINQPIAAITSYSEGGIMRLQGKEQADTDSCIELLEKIHKQSTRAGEVVHRIRGLLKRREAVMVDVNILTLVEESISLLRLELARREIQINTQIKGEPFFITADRVGLLQVLINLIKNSLDAIAESDNARSGKINIELDFKEYQVNVSIIDNGPGLAMDSDTLMATFYTTKMDGLGLGLAICREVISNHDGHFLLSNRDDGVLGCVATLNLKKRGSEVPIEV

SEQ ID NO: 2 - protein, 203 aa, Shewanella halifaxensis
MQQQINGPVYLVDDDEAIIDSIDFLMEGYGYKLNSFNCGDRFLAEVDLTQAGCVILDARMPGLTGPQVQQLLSDAKSPLAVIFLTGHGDVPMAVDAFKNGAFDFFQKPVPGSLLSQSIAKGLTYSIDQHLKRTNQALIDTLSEREAQIFQLVIAGNTNKQMANELCVAIRTIEVHRSKLMTKLGVNNLAELVKLAPLLAHKSE

(19) United States
(12) Patent Application Publication
(10) Pub. No.: US 2018/0363025 A1
(43) Pub. Date: Dec. 20, 2018

(54) THIOSULFATE BIOSENSOR FOR GUT INFLAMMATION

(71) Applicant: William Marsh Rice University, Houston, TX (US)

(72) Inventors: Jeffrey J. TABOR, Houston, TX (US); Kristina N.M. DAEFFLER, US, TX (US); Ravi SHETH, Houston, TX (US)

(21) Appl. No.: 15/760,931

(22) PCT Filed: Sep. 16, 2016
(86) PCT No.: PCT/US2016/052138

§ 371 Date: Mar. 16, 2018

Related U.S. Application Data
(60) Provisional application No. 62/220,126, filed on Sep. 17, 2015.

(51) Int. Cl. C12Q 1/48; C12N 9/12; C07K 14/195; A61K 35/741; C12Q 1/6897

(52) U.S. Cl. CPC C12Q 1/485; C12N 9/12; A61K 2035/115; A61K 35/741; C12Q 1/6897; C07K 14/195

(57) ABSTRACT

Bacteria, systems and methods comprising two-component sensor systems used to detect thiosulfate are described. The systems can be applied to monitoring gut bacteria.

### PRIOR RELATED APPLICATIONS

This application claims priority to 62/220,126, filed Sep. 17, 2015 and incorporated by reference herein in its entirety for all purposes.

### FEDERALLY SPONSORED RESEARCH STATEMENT

This invention was made with government support under N00014-14-1-0487 awarded by the Office of Naval Research. The government has certain rights in the invention.

### FIELD OF THE DISCLOSURE

The invention is microbes, systems and methods comprising two-component sensor systems from bacteria in order to detect thiosulfate.

### BACKGROUND OF THE DISCLOSURE

A two-component regulatory system serves as a basic stimulus-response coupling mechanism to allow organisms to sense and respond to changes in many different environmental conditions. Such systems typically consist of a membrane-bound histidine kinase that senses a specific environmental stimulus and a corresponding response regulator that mediates the cellular response, mostly through differential expression of target genes.

Two-component signal transduction systems enable bacteria to sense, respond, and adapt to a wide range of environments, stressors, and growth conditions. Some bacteria can contain up to as many as 200 two-component sensor systems. These pathways have been adapted to respond to a wide variety of stimuli, including nutrients, cellular redox state, changes in osmolarity, quorum signals, antibiotics, temperature, chemoattractants, pH and more.

As an example, in Escherichia coli the EnvZ/OmpR osmoregulation system controls the differential expression of the outer membrane porin proteins OmpF and OmpC.

Two-component systems accomplish signal transduction through the phosphorylation of a response regulator (RR) by a histidine kinase (HK). Histidine kinases are frequently homodimeric transmembrane proteins containing a histidine phosphotransfer domain and an ATP binding domain, but HKs can be cytoplasmic. Response regulators may consist only of a receiver domain, but usually are multi-domain proteins with a receiver domain and at least one effector or output domain, often involved in DNA binding.

Upon detecting a particular change in the extracellular environment, the HK performs an autophosphorylation reaction, transferring a phosphoryl group from adenosine triphosphate (ATP) to a specific histidine residue. The cognate response regulator (RR) then catalyzes the transfer of the phosphoryl group to an aspartate residue on the response regulator's receiver domain. This typically triggers a conformational change that activates the RR's effector domain, which in turn produces the cellular response to the signal. Frequently, the effector domain is a DNA binding domain that enables the RR to stimulate (or repress) transcription from one or more target promoters and expression of one or more target genes.

Many HKs are bifunctional and possess phosphatase activity against their cognate response regulators, so that their signaling output reflects a balance between their kinase and phosphatase activities. Many phosphorylated RRs (RR˜Ps) also auto-dephosphorylate. The relatively labile phosphoaspartate can also be hydrolyzed non-enzymatically. The overall level of phosphorylation of the RR population in the cell ultimately controls the activity of the two-component system.

It is possible to identify two-component systems from bacterial genome sequences by computational methods, such as homology and/or domain searching. However, such systems typically have unknown inputs and outputs. In the case of two-component systems wherein the RR has a DNA binding domain, this means that a computationally identified two-component system frequently controls expression of unknown output genes. Because both key pieces of information are lacking, and because the microbes that contain them are often un-culturable and/or difficult to genetically manipulate in the laboratory, it is very difficult to identify the inputs that they sense. Therefore, while sensor kinase systems have tremendous medical, industrial and basic research applications, they have not yet to be fully exploited.

This application provides a thiosulfate biosensor based on a newly identified thiosulfate sensing two-component system with transcriptional output and also provides various applications for the biosensor. One particular application relates to diagnosing and/or treating gut pathologies.

### SUMMARY OF THE DISCLOSURE

Thiosulfate is a potential respiratory electron acceptor for bacteria that live in anoxic environments or at the anoxic/oxic interface. The ability to respire thiosulfate is conferred by the enzyme thiosulfate reductase which catalyzes the reaction:

S₂O₃²⁻+2H⁺+2e⁻→HS⁻+HSO₃⁻

Thiosulfate is a significant intermediate in the sulfur cycle of anoxic marine and freshwater sediments, where it is involved in reduction, oxidation, and disproportionation pathways. The net effect of these reactions is to keep the thiosulfate concentration in these environments relatively low (submicromolar to about 10 μM). Thiosulfate reduction in sediments is primarily carried out by sulfate-reducing bacteria. Indeed, sulfate-reducing bacteria preferentially use thiosulfate over sulfate as an electron acceptor. In sulfate-reducing bacteria, the sulfite produced in the thiosulfate reductase reaction is further reduced to sulfide by sulfite reductase. Sulfite reduction is an energy-yielding reaction that is also the final step in sulfate respiration. Certain sulfate-reducing bacteria are able to grow by thiosulfate disproportionation to sulfide and sulfate, a pathway in which the first step is proposed to be thiosulfate reduction by thiosulfate reductase. However, thiosulfate reductase activity is not restricted to sulfate-reducing bacteria but can be found in other types of environmentally abundant bacteria, such as Shewanella species.

Thiosulfate can also be found in the mammalian gut. Bacteria present in the lumen of the large intestine produce sulfide by reduction of dietary sulfate and sulfite, by fermentation of sulfur-containing amino acids, and by metabolism of sulfated mucopolysaccharides. To protect the animal from the toxic effects of this microbially produced sulfide, mitochondria in the colonic mucosa catalyze the oxidation of sulfide to thiosulfate. The thiosulfate produced is then available as a respiratory substrate for colonic bacteria. The ability of certain enteric pathogens to produce sulfide from thiosulfate has been known for almost one hundred years and is the basis of some commercial tests used for strain differentiation in clinical diagnostic laboratories. Genera of enteric bacteria that typically reduce thiosulfate include Salmonella, Proteus, Citrobacter, and Edwardsiella.

The produced thiosulfate can subsequently be used as a reactive oxygen species scavenger during inflammation to prevent tissue damage. Indeed, exogenously administered sodium thiosulfate has been shown to have anti-inflammatory and cardioprotective effects through attenuation of oxidative stress in the body. However, in the gut, the oxidation product of thiosulfate, tetrathionate, is a high-energy terminal electron acceptor usable by pathogenic bacteria such as Salmonella. These bacteria can thereby gain an advantage in inflammatory conditions, which leads to major blooms of Enterobacteria and a dramatic alteration of the gut microbial population, known as dysbiosis.

An ability to detect and measure physiological concentrations of thiosulfate at the site of inflammation would provide a novel measure of gut health. A bacterial sensor is an ideal solution because it can pass through the gut as a non-invasive observer while providing a readout of gut health that does not require removal of human tissue. Also, many usable bacterial strains (probiotics) are already approved for human consumption, allowing in vivo diagnostics. Alternatively, the bacteria can be exposed to gastrointestinal or fecal samples in ex vivo bench top assays, or cell extracts or purified proteins can be used in in vitro methods.

This disclosure provides the first demonstration of a genetically engineered and encoded thiosulfate biosensor, as well as the first use of such a biosensor as a non-invasive bacterial diagnostic of gut health. The genes for thiosulfate utilization by bacteria were known, but previously there were no characterized molecular sensors, nor transcriptional regulators of these genes. Therefore, both characterization and implementation of the sensor in a suitable bacterial host are novel.

We have characterized and harnessed a naturally occurring thiosulfate sensor from a marine bacterium, altered its genetic control elements and output response, and demonstrated thiosulfate-sensing activity in vitro in a gut-adapted bacterium with high specificity for the desired ligand. Engineered bacteria that sense thiosulfate, an important metabolite involved in gut homeostasis, will serve as a diagnostic of healthy or diseased gut conditions and enable the spatial and temporal release of therapeutic agents at the site of dysbiosis.

The thiosulfate sensor was identified by bioinformatics analysis based on homology to a sensor for tetrathionate, a chemically similar molecule, and proximity to a putative thiosulfate reductase. The SK: Shal_3128, from Shewanella halifaxensis and its cognate RR: Shal_3129, were cloned onto E. coli expression plasmids under the tunable expression of inducible promoters. The intergenic region upstream of the putative thiosulfate reductase was cloned as the predicted output promoter of the SK/RR pair and sensitivity to thiosulfate was determined by measuring superfolder green fluorescent protein (GFP) output from the promoter in the presence and absence of thiosulfate. Once thiosulfate was confirmed as the input for this sensor, the expression of both proteins were tuned to give the highest dynamic range.

The range of thiosulfate concentrations found in healthy and diseased mammalian guts is currently unknown, but studies are planned to obtain this information. The sensitive range of the developed sensor may therefore not be appropriate for measuring these concentrations because Shewanella have not evolved to survive in the gut environment. Sensor sensitivity can be tuned, however, to enhance or weaken thiosulfate-induced activation of the sensor using mutagenesis, optimizing codon usage, enhancers, stabilizers, and the like, or homologous sensors can be mined and screened that may naturally have evolved different sensitivities.

The developed thiosulfate sensing system has undergone one round of optimization and subsequent validation in mouse models. However, it can be further optimized for enhanced performance in in vivo human use. The resulting sensor will then be tested in vivo in healthy and diseased mouse models to determine sensing capabilities in a mammalian gut before undergoing testing in human studies. Further optimization of the bacterial diagnostic may be performed to enhance detection capabilities at physiologically relevant thiosulfate concentrations, if needed.

In more detail, the invention includes one or more of the following embodiments, in any combination(s) thereof:

Genetically engineered bacteria, said bacteria expressing or overexpressing:

| | |
|---|---|
| a) | a two-component sensor system (TCS) comprising: |
| i) | a thiosulfate-sensing sensor kinase (SK) gene comprising a ligand binding domain |
| | operably coupled to a kinase domain; and, |
| ii) | a cognate response regulator (RR) gene comprising a receiver domain operably coupled |
| | to an DNA binding domain (DBD); |
| b) | an output promoter comprising a DNA binding site that binds said DBD and is operably |
| | coupled to a reporter gene. |

Genetically engineered bacteria, said bacteria comprising:

| | |
|---|---|
| a) | a heterologous thiosulfate sensor system comprising: |
| | i) a thiosulfate-sensing sensor kinase (SK) gene comprising a ligand binding domain |
| | operably coupled to a kinase domain; and, |
| | ii) a cognate response regulator (RR) gene comprising a receiver domain operably coupled |
| | to an DNA binding domain (DBD); |
| b) | a DNA binding site that binds said DBD that is operably coupled to either a reporter gene |
| | or a therapeutic protein gene. |

Any bacteria herein described, wherein said RR gene is rewired such that said receiver domain
is operably coupled to a heterologous DBD from another gene.
Any bacteria herein described, wherein said SK gene or said RR gene or both genes are
encoded on an expression vector.
Any bacteria herein described, wherein said SK gene or said RR gene or both genes are
encoded on an inducible expression vector.
Any bacteria herein described, wherein said SK gene or said RR gene or both genes are
encoded on an constitutive expression vector.
Any bacteria herein described, wherein said SK gene or said RR gene or both genes
integrated into a chromosome of said bacteria.
Any bacteria herein described, wherein said SK gene and said RR gene are encoded in a
single operon.
Any bacteria herein described, wherein said output promoter and said reporter gene are
encoded on a plasmid.
Any bacteria herein described, wherein said output promoter and said reporter gene are
integrated into a chromosome of said bacteria.
Any bacteria herein described, wherein said SK gene and said RR gene are from
Shewanella halifaxensis.
Any bacteria herein described, comprising SEQ ID NO. 1 and No. 2 or any other sequences
herein described.
Any bacteria herein described, wherein said reporter gene encodes a fluorescent protein, such
as green fluorescent protein, red fluorescent protein, far red fluorescent protein, blue
fluorescent protein, orange fluorescent protein, yellow fluorescent protein, mCHERRY,
mORANGE, mCITRINE, VENUS, YPET, EMERALD, or CERULEAN.
Any bacteria herein described, wherein said reporter gene encodes a colorimetric protein such
as β-galactosidase, β-glucuronidase, or alkaline phosphatase.
Any bacteria herein described, wherein said reporter gene encodes a luminescent protein such
as bacterial luciferase, firefly luciferase, or click beetle luciferase
Any bacteria herein described, wherein said reporter gene encodes a ‘barcoded’ messenger
RNA containing a unique nucleotide sequence enabling identification and quantitation via
methods such as quantitative RT-PCR or RNA-seq
Any bacteria herein described, wherein said bacteria is probiotic for use in humans.
A method of detecting thiosulfate, comprising:
i) combining a test sample with a bacteria herein described; and,
ii) measuring expression of said reporter gene, wherein a change in a level of expression of
said reporter gene as compared to a control sample lacking thiosulfate indicates that said test
sample contains thiosulfate.
A method of detecting excess thiosulfate levels in a patient, comprising i) administering a
bacteria herein described to a patient, ii) collecting a stool sample from said patient; iii)
measuring expression of said reporter gene in said stool sample, wherein a change in level of
expression of said reporter gene over a normal level in a normal patient indicates that said
patient has excess thiosulfate.
A method of measuring thiosulfate levels in a patient, comprising:

| | |
|---|---|
| a) | combining a gut or fecal sample with a thiosulfate reporter bacteria comprising: |
| i) | a thiosulfate-sensing sensor kinase (SK) gene encoding an SK protein comprising a |
| | ligand binding domain that binds thiosulfate and activates a kinase domain, |
| ii) | a cognate RR gene encoding an RR protein comprising a receiver domain operably |
| | coupled to an DNA binding domain (DBD), wherein said cognate RR protein is activated |
| | by said activated kinase domain phosphorylating said receiver domain, and |
| iii) | a DNA binding site that binds said DBD of said cognate activated RR protein, wherein |
| | said DNA binding site is operably coupled to a reporter gene; |
| b) | measuring expression of said reporter gene; and, |
| c) | correlating a measured level of reporter gene expression with a level of thiosulfate using a |
| | standard curve. |

A treatment method, comprising administering a bacteria herein described to a patient having
excess thiosulfate, wherein said DBD is operably coupled to a therapeutic protein (or the genes
encoding both components are operably coupled or fused).
A treatment method, comprising administering a bacteria herein described to a patient having
excess thiosulfate, wherein said DBD is operably coupled to a therapeutic protein that reduces
inflammation.
A method of screening for gut inflammation, comprising i) administering a bacteria herein
described to a patient, ii) collecting a gut or stool sample from said patient, and iii) measuring
activity of said reporter gene in said gut or stool sample, wherein a change in reporter gene
expression over a normal level in a normal patient indicates that said patient has gut
inflammation.

There are a great variety of reporter genes that can be used herein, and GFP is only one convenient reporter. Other fluorescent proteins include, but are not limited to red fluorescent protein, far red fluorescent protein, blue fluorescent protein, orange fluorescent protein, yellow fluorescent protein, mCHERRY, tdTOMATO, mORANGE, mCITRINE, VENUS, YPET, EMERALD, mNEONGREEN and CERULEAN. A great many others are available, see e.g., nic.ucsfedu/dokuwiki/doku.php?id=fluorescent_proteins, incorporated by reference herein in its entirety for all purposes.

The amount or activity of the reporter protein produced is taken as a proxy for the cellular response to the target. Ideal reporter proteins are easy to detect and quantify (preferably noninvasively), highly sensitive and, ideally, not present in the native organism. They can be set up to detect either gene activation or deactivation. Several currently popular reporter proteins and their characteristics are listed in TABLE 1. For in vivo use, a longer lasting reporter signal (8-12 hrs) may be preferred, such that signal can still be detected in stool samples.

TABLE 1
Common spectroscopically active reporter proteins and their detection

| | Reporter | | | | | |
|---|---|---|---|---|---|---|
| Reporter protein | genes | Origin | Substrate | Detection method | Comments | Refs |
| Bacterial | luxAB* or | Bioluminescent | O₂, FMNH₂ and | Bioluminescence | Requires O₂; aldehyde | 94, 95 |
| luciferase | luxCDABE | bacteria* | long-chain aldehydes | | addition is required if | |
| | | | | | only luxAB is used | |
| Firefly luciferase | lucFF | Firefly (Photinus pyralis) | O₂, ATP and luciferin | Bioluminescence | Requires O₂ | 96 |
| Click beetle | lucGR | Click beetle (Pyrophorus | O₂, ATP and pholasin | Bioluminescence | Requires O₂ | 97 |
| luciferase | | plagiophthalamus) | | | | |
| Renilla luciferase | Rluc | Renilla reniformis | Coelenterazine and | Bioluminescence | Requires O₂ | 98 |
| | | | Ca², | | | |
| β-Galactosidase | lacZ | Escherichia coli | Galactopyranosides* | Chemiluminescence, | External substrate | 1 |
| | | | | colorimetry, | addition (may require | |
| | | | | electrochemistry and | cell permeabilization) | |
| | | | | fluorescence | | |
| Fluorescent | gfp. etc. | Aequorea victoria and | N/A | Fluorescence | O₂ is required for | 99-101 |
| proteins | | additional marine | | | maturation; different | |
| | | invertebrates | | | colour varieties exist | |
| Spheroidene | crtA | Rhodovulum | Spheroidene | Colorimetry | None | 102 |
| monooxygenase | | sulfulophilum | | | | |
| Infrared | Various | Bacteriophytochrome | N/A | Fluorescence | None | 103 |
| fluorescent | | family | | | | |
| proteins | | | | | | |
| FMN-based | Various | Engineered from | None | Fluorescence | Functional in both oxic | 104 |
| fluorescent | | Bacillus subtilis and | | | and anoxic conditions: | |
| proteins | | Pseudomonus putida | | | requires endogenous | |
| | | | | | FMN | |

N/A, not applicable.
* Most commonly used species include Altivibrio fischeri (also known as Vibrio fischeri). Vibrio harveyi and Photor habdus luminescence.
‡For example, O-nitrophenyl-β-o-galactoside (ONPG), 5-bromo-4-chloro-3-indolyl-β-o-galactopyranoside (X-gal), 4-methylumbelliferyl-β-o-galactopyranoside, 4-aminophenyl-β-o-galactopyranoside and o-luciferin-O-β-galactopyranoside.

Using the amount of reporter gene as a readout, and using standard high throughput screening methods, such as fluorimetry or flow-cytometry, we can screen a novel TCS against virtually any chemical or physical input, and very easily measure those chemicals that it senses, using standard, high throughput laboratory assays. This method can thus be used to identify other thiosulfate sensor genes for use herein.

Initial experiments proceeded in E. coli for convenience, but the addition of genes to bacteria is of nearly universal applicability, so it will be possible to use a wide variety of organisms with the selection of suitable vectors for same. Various probiotic Lactobacillus and Bifidobacterium may be particularly suitable for in vivo use. Furthermore, a number of databases include vector information and/or a repository of vectors. See e.g., Addgene.org, which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are often available from colleagues.

Once an exemplary sequence is obtained, e.g., in E. coli, which is completely sequenced and which is the workhorse of genetic engineering and bioproduction, many additional examples proteins of similar activity can be identified by BLAST search or database search. The OMIN database is also a good resource for searching human proteins and has links to the sequences. Further, every protein record is linked to a gene record, making it easy to design genome insertion vectors. Many of the needed sequences are already available in vectors, and can often be obtained from cell depositories or from the researchers who cloned them. But, if necessary, new clones can be prepared based on available sequence information using gene synthesis or PCR techniques. Thus, it should be easily possible to obtain all of the needed sequences.

Understanding the inherent degeneracy of the genetic code allows one of ordinary skill in the art to design multiple sequences that encode the same amino acid sequence. NCBI® provides codon usage databases for optimizing DNA sequences for protein expression in various species. Using such databases, a gene or cDNA may be “optimized” for expression in E. coli, Lactobacillus, Bifidobacterium, mice, humans, or other species using the codon bias for the species in which the gene will be expressed.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence).

Alignments are performed using BLAST homology alignment as described by Tatusova T A & Madden T L (1999) FEMS Microbiol. Lett. 174:247-250. The default parameters were used, except the filters were turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 1 1 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=1 1 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (ncbi.nlm.nih.gov/BLAST/). “Positives” includes conservative amino acid changes in addition to identities.

As used herein, a “two component system” or “two component sensor system” or “TCS” is understood to be a two protein system including a sensor kinase and a response regulator, wherein the sensor kinase when bound to its cognate ligand, activates the response regulator which then activates the expression of relevant downstream proteins.

“Cognate” refers to two components systems that function together, such as e.g., a SK will bind to its cognate RR and activate it. The SK and RR are thus cognate, meaning they function together or are functionally related or connected.

As used herein, a “sensor kinase” or “SK” is a protein understood to have a ligand binding domain (“LBD”) operably coupled to a kinase domain (“KD”), such that when the LBD binds its cognate ligand, the kinase is activated.

As used herein, a response regulator typically has a “receiver” or “REC” domain that is activated by the active kinase of the TCS. Typically the REC domain is operably coupled to a DNA binding domain or DBD, which thus can bind to and turn on relevant downstream protein expression.

As used herein, a “rewired” RR means that either the gene output of the native two-component system has been changed to instead provide a desirable output such as a reporter or therapeutic, or that the DBD of the RR has been modularly swapped for the DBD of another RR, which is then used to control expression of a desirable output such as a reporter or therapeutic.

As used herein, a “heterologous DBD” means a DBD that comes from another protein, not the response regulator that the REC domain comes from. Typically, the DBD then binds to the DNA it is targeted to, which is itself coupled to a reporter gene that can easily be detected. Alternatively, a therapeutic protein could be expressed, e.g. a bacterial toxin for targeted treatment of inflammation.

As used herein, reference to cells, bacteria, microbes, microorganisms and like is understood to include progeny thereof having the same genetic modifications. It is also understood that all progeny may not be precisely identical in DNA content, due to deliberate or inadvertent mutations that have been added to the parent. Mutant progeny that have the same function or biological activity as screened for in the originally transformed cell are included. Where distinct designations are intended, it will be clear from the context.

The terms “operably associated” or “operably linked,” as used herein, refer to functionally coupled nucleic acid or amino acid sequences.

As used herein “recombinant” or “engineered” is relating to, derived from, or containing genetically engineered material. In other words, the genome was intentionally manipulated in some way.

“Reduced activity” or “inactivation” is defined herein to be at least a 75% reduction in protein activity, as compared with an appropriate control species. Preferably, at least 80, 85, 90, 95% reduction in activity is attained, and in the most preferred embodiment, the activity is eliminated (100%, aka a “knock-out” or “null” mutants). Proteins can be inactivated with inhibitors, by mutation, or by suppression of expression or translation, and the like. Use of a frame shift mutation, early stop codon, point mutations of critical residues, or deletions or insertions, and the like, can completely inactivate (100%) gene product by completely preventing transcription and/or translation of active protein.

“Overexpression” or “overexpressed” is defined herein to be at least 150% of protein activity as compared with an appropriate control species, and preferably 200, 500, 1000%) or more, or any activity in a species that otherwise lacks the activity. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or upregulating the endogenous gene, and the like.

The term “endogenous” or “native” means that a gene originated from the species in question, without regard to subspecies or strain, although that gene may be naturally or intentionally mutated, or placed under the control of a promoter that results in overexpression or controlled expression of said gene. Thus, genes from Clostridia would not be endogenous to Escherichia, but a plasmid expressing a gene from E. coli would be considered to be endogenous to any species of Escherichia, even though it may now be overexpressed. A “wild type” sequence is a functional gene unchanged from its host species, e.g, is naturally occurring.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, background mutations that do not effect the invention, and the like.

The following abbreviations are used herein:

| ABBREVIATION | TERM |
|---|---|
| ATP | Adenosine triphosphate |
| CmR | Chloramphenicol resistance gene |
| CRP | cAMP receptor protein |
| DBD | DNA binding domain |
| DSS | Dextran Sodium Sulfate |
| GFP | Green fluorescent protein |
| HK | Histidine kinases |
| HPT | Histidine phosphotransferase |
| HPt | His-containing phosphotransfer |
| IPTG | Isopropyl β-D-1-thiogalactopyranoside |
| KD | Kinase domain |
| LBD | Ligand binding domain |
| RBS | Ribosome binding site |
| REC | Receiver domain |
| RR | Response regulator |
| SK | Sensor kinase |
| TCS | Two component sensor system, including a KD |
| | and a RR |
| TEA | Terminal electron acceptor |
| TetR | Tetracyline repressor protein |

### BRIEF DESCRIPTION OF FIGURES

FIG. 1. Schematic of method.

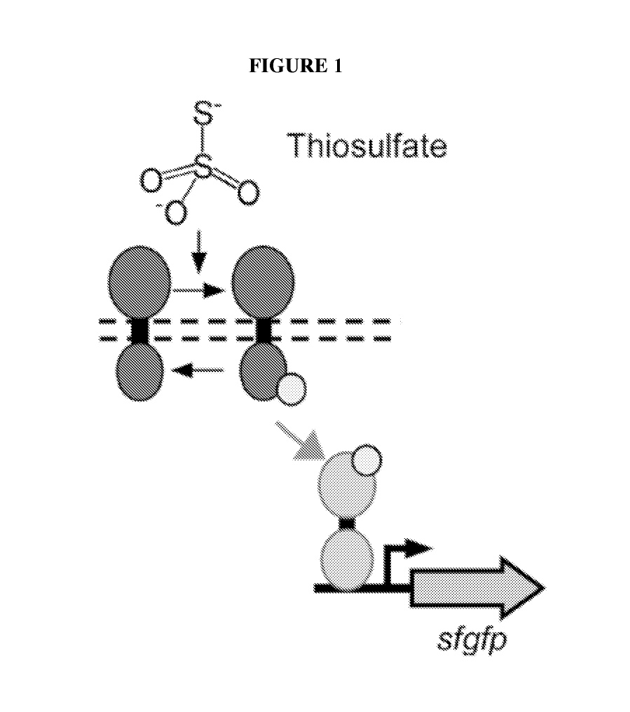

FIG. 2 Plasmids used herein.

FIG. 3A-F. Characterization of the thiosulfate-sensing proteins: Shal_3128/9. (3A) The S. halifaxensis genomic region containing the thiosulfate reductase operon (Shal_3125-7) and neighboring thiosulfate-sensing TCS (Shal_3128/9). (3B) Plasmid design of the inducible thiosulfate sensor. (3C) Controls indicating that thiosulfate sensing proceeds through the canonical route. (3D) Selectivity of Shal_3128/9 to thiosulfate over other terminal electron acceptors. All TEAs were tested at 10 mM concentration. (3E) Ligand competition assay. Sensor performance at 5 mM thiosulfate was tested in the presence of 10 mM other TEAs, and a decrease in response indicates inhibition by the added ligand. (3F) Inhibition curve of tetrathionate (filled circles) and sulfite (open squares) in the presence of 5 mM thiosulfate.

FIG. 4A-D. Sensor optimization for thiosulfate detection in the gut. (4A) Changes made between the initial characterization constructs and in vivo optimized sensor. (4B) Layout of sensor components on the final two plasmid expression system. (4C) Normalized dose-response behavior of thiosulfate sensors. Shown is the original, inducible, BW28357 strain grown aerobically (closed circles), and the selected constitutive promoter strain in Nissle grown aerobically (closed squares) and anaerobically (open squares). A shift in half-maximal response indicates sensitivity to oxygen. (4D) Dynamic range of optimized thiosulfate sensors. GFP output is shown in the absence (grey bars) and presence of 5 mM thiosulfate or 1 mM tetrathionate, respectively (black bars). 3% DSS, the chemical used to initiate colonic inflammation, does not activate or inhibit signaling of either sensor.

FIG. 5A-D. In vivo measurement of thiosulfate and tetrathionate in healthy and inflamed guts. (5A) Experimental design. 6-8 week old C57BL/6 mice were given water with or without 3% DSS for 5 days before oral gavage (via feeding tube) with sensor bacteria. After 6 hours samples were collected from the mice, processed, and analyzed by flow cytometry to measure GFP production. Mice were gavaged with 10⁹ bacteria of the (5B) thiosulfate sensor or the (5C) thiosulfate sensor negative control (D57A) *p<0.005 and **p<0.001. Sensor output is determined by fluorescence. (5D) A correlation of histology score and thiosulfate sensor output from fecal samples is observed indicating thiosulfate is a biomarker of inflammation.

### DETAILED DESCRIPTION

The thiosulfate sensor system exemplified herein consists of two proteins, a thiosulfate-sensing sensor kinase (SK)—Shal_3128, from Shewanella halifaxensis and its cognate response regulator (RR)—Shal_3129. Binding of thiosulfate to the sensor domain of Shal_3128 results in activation of Shal_3129 through transfer of a phosphate from the SK to the RR. The activated RRs can then bind to regulatory sites on a genetically encoded promoter and facilitate enhanced transcription of a downstream gene. The output of this sensor can either be a reporter gene such as a fluorescent protein for use as a diagnostic or a protein therapeutic for direct treatment of diseases, e.g., a inflammation inhibitor or antibiotic.

Amino acid sequence of Shal_3128 (SK) (SEQ ID
NO. 1):
MSRLLLCICVLLFSSVAWSKPQQFYVGVLANWGHQQAVERWTPMMEYLNE
HVPDAEFHVYPGNFKALNLAMELGQIQFIITNPGQYLYLSNQYPLSWLAT
MRSKRHDGTTSAIGSAIIVRADSDYRTLYDLKGKVVAASDPHALGGYQAT
VGLMHSLGMDPDTFFGETKFLGFPLDPLLYQVRDGNVDAAITPLCTLEDM
VARGVLKSSDFRVLNPSRPDGVECQCSTTLYPNWSFAATESVSTELSKEI
TQALLELPSDSPAAIKAQLTGWTSPISQLAVIKLFKELHVKTPDSSRWEA
VKKWLEENRHWGILSVLVFIIATLYHLWIEYRFHQKSSSLIESERQLKQQ
AVALERLQSASIVGEIGAGLAHEINQPIAAITSYSEGGIMRLQGKEQADT
DSCIELLEKIHKQSTRAGEVVHRIRGLLKRREAVMVDVNILTLVEESISL
LRLELARREIQINTQIKGEPFFITADRVGLLQVLINLIKNSLDAIAESDN
ARSGKINIELDFKEYQVNVSIIDNGPGLAMDSDTLMATFYTTKMDGLGLG
LAICREVISNHDGHFLLSNRDDGVLGCVATLNLKKRGSEVPIEV*

Additional proteins that can substitute for SEQ ID NO. 1 have been identified by homology search (a few of which are listed below along with amino acid identity level), and functionality can be confirmed as described herein.

| | |
|---|---|
| Shewanella fidelis WP_028769121.1 | 558/589(94%) |
| Shewanella piezotolerans, WP_020911409.1 | 504/581(86%) |
| Ferrimonas senticii, WP_035387522.1 | 462/569(81%) |
| Ferrimonas kyonanensis, WP_035416355.1 | 458/571(80%) |
| Shewanella colwelliana, WP_028762598.1 | 427/599(71%) |

Amino acid sequence of Shal_3129 (RR) (SEQ ID
NO 2):
MQQQINGPVYLVDDDEMIDSIDFLMEGYGYKLNSFNCGDRFLAEVDLTQA
GCVILDARMPGLTGPQVQQLLSDAKSPLAVIFLTGHGDVPMAVDAFKNGA
FDFFQKPVPGSLLSQSIAKGLTYSIDQHLKRTNQALIDTLSEREAQIFQL
VIAGNTNKQMANELCVAIRTIEVHRSKLMTKLGVNNLAELVKLAPLLAHK
SE*

Additional proteins that can substitute for SEQ ID NO. 2 have been identified by homology search (a few of which are listed below along with amino acid identity level), and functionality can be confirmed as described herein.

| | |
|---|---|
| [Shewanella pealeana] WP_012156262.1 | 192/202(95%) |
| [Shewanella fidelis] WP_028769122.1 | 186/203(91%) |
| [Shewanella waksmanii] WP_028774189.1 | 167/198(84%) |
| [Shewanella piezotolerans] WP_020911408.1\| | 171/198(86%) |
| [Ferrimonas senticii] WP_028118108. | 156/190(82%) |
| [Ferrimonas kyonanensis] WP_028114599.1 | 147/191(76%) |
| [Ferrimonas futtsuensis] WP_028109475.1 | 148/198(74%) |

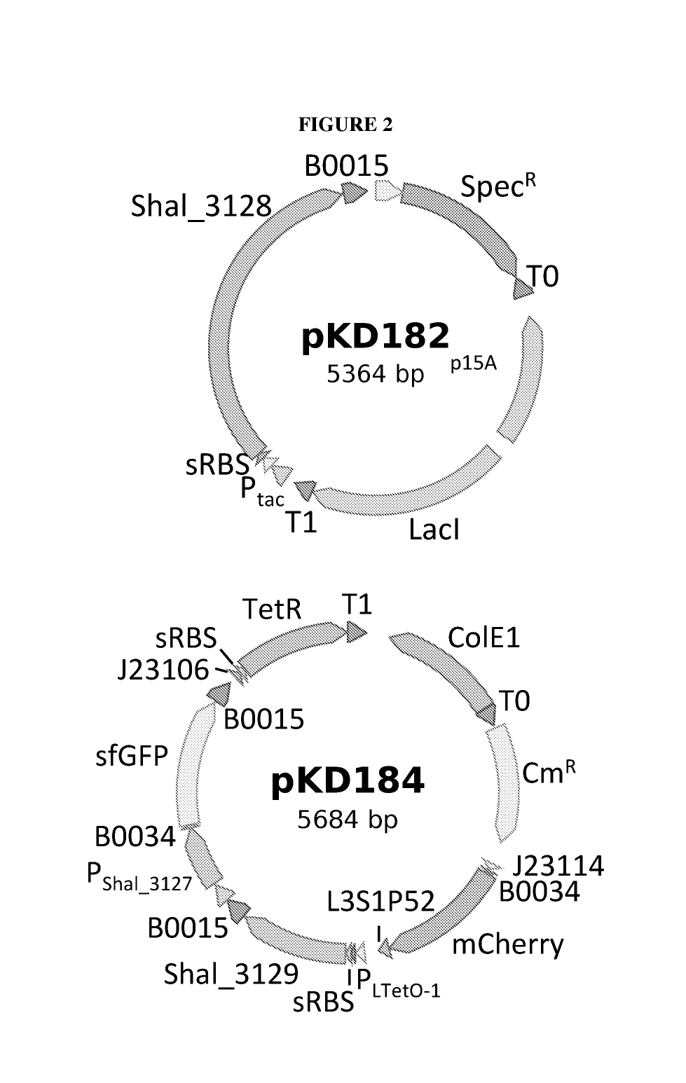

FIG. 2 displays plasmids used herein for in vitro characterization of the TCS. The sensor kinase was expressed from a p15A medium-copy expression vector with spectinomycin resistance. Production of Shal_3128 was driven by the P_{tac} promoter and a designed synthetic RBS (predicted strength 1,000), which was induced by IPTG and regulated by constitutively expressed LacI. Transcription of Shal_3128, LacI, and Spec^{R} was terminated by the B0015, T1, and T0 terminators, respectively.

The response regulator and rewired output promoter driving GFP production were on a ColE1 high-copy expression vector with chloramphenicol resistance. Shal_3129 production was regulated by constitutively expressed TetR at the P_{LTetO-1} aTc-inducible promoter with a synthetic RBS (predicted strength 1,000). The output promoter (P_{Shal}_{_}₃₁₂₇) was the 342 bp intergenic region of the vector, upstream of the thiosulfate reductase genes (Shal_3125-3127). Production of sfGFP was regulated by output of the P_{Shal}_{_}₃₁₂₇ promoter using the B0034 RBS, which provides a visual readout of Shal_3128/9 activity. Transcription of Shal_3129, sfGFP, TetR, and Cm^{R} was terminated by the B0015, B0015, T1, and T0 terminators, respectively.

Both plasmids were transformed into the BW28357 E. coli strain, available from The Coli Genetic Stock Center (CGSC#: 7991, F−, Δ(araD-araB)567, ΔlacZ4787(TrnB-3); lambda⁻, Δ(rhaD-rhaB)568, hsdR514).

In the above proof of concept experiments, the wild type SK and RR were used because the downstream output promoter was known. However, it is possible to rewire the RR to have the DBD from another protein, and to modify the reporter gene to respond to that heterologous DBD. Indeed, such methods may be preferred because it is another point at which the system can be improved for higher sensitivity. This method might also be appropriate in switching to a very different host species, e.g., gram negative to gram positive, where the original RR might be ineffective or less effective. We have already confirmed that it is possible to move an RR from a gram negative species into a gram positive species by this method.

All of these expression components are exemplary only, however, and there are thousands of suitable components to choose from.

An exemplary growth and assay protocol follows, but the details can be changed:
Overnight pre-culture (˜13 hours) in LB+Cm/Spec
Dilute to OD₆₀₀=0.02 in M9+0.4% glycerol
Grow 3 hours to OD₆₀₀=˜0.3
Dilute to OD₆₀₀=0.0001 in M9+0.4% glycerol
Add IPTG and desired thiosulfate (5 mM for max response)
Grow shaking at 37° C.˜7 hours to OD₆₀₀=˜0.3
Put on ice, measure OD, measure fluorescence by flow cytometry (FL1=700, FL3=850)

Although a single gene pair was exemplified herein (albeit with at least two promoters each) in two host species, there are two features that indicate broad applicability of the invention. The first feature is tunability, which is particularly important for sensing thiosulfates because the biological ranges for levels of thiosulfate in humans has not been studied. Because this system is tunable, once that range is known the sensor can be easily tuned to sense and provide output at the needed levels.

The second feature piggybacks on the tunability function but also relies on the fact that the inventors have engineered and characterized a suite of DBD, promoters, and reporters for use in this system (described in 62/157,293). When combined, these features allow the inventors to transfer the system to a broad range of microbial species and strains.

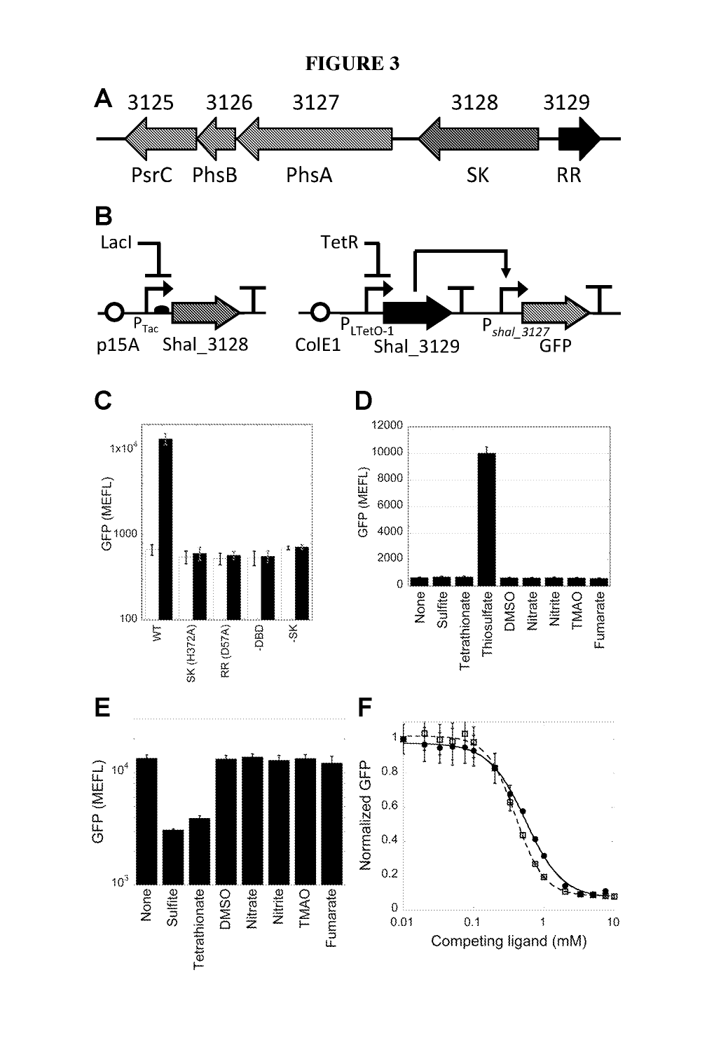

FIG. 3 shows the characterization of the thiosulfate-sensing TCS developed herein. When the catalytic histidine in the SK and the phospho-accepting aspartate in the RR are mutated, sensor function is lost indicating sensing proceeds through the canonical signaling pathway of TCSs (FIG. 3C). Also, if Shal_3128 is excluded or the DBD is removed from the RR, no thiosulfate response is observed, eliminating cross-talk from endogenous TCSs as a source of signaling. All TEAs were tested at 10 mM concentration and as can be seen in FIG. 3D, only thiosulfate effectively stimulated production of reporter protein GFP. In a ligand competition assay, a decrease in response indicates inhibition by the added ligand. As can be seen in FIG. 3E, thiosulfate competed all ligands effectively, although the similar molecules sulfite and tetrathionate were able to provide a modest level of competition at concentrations higher than expected in physiological conditions.

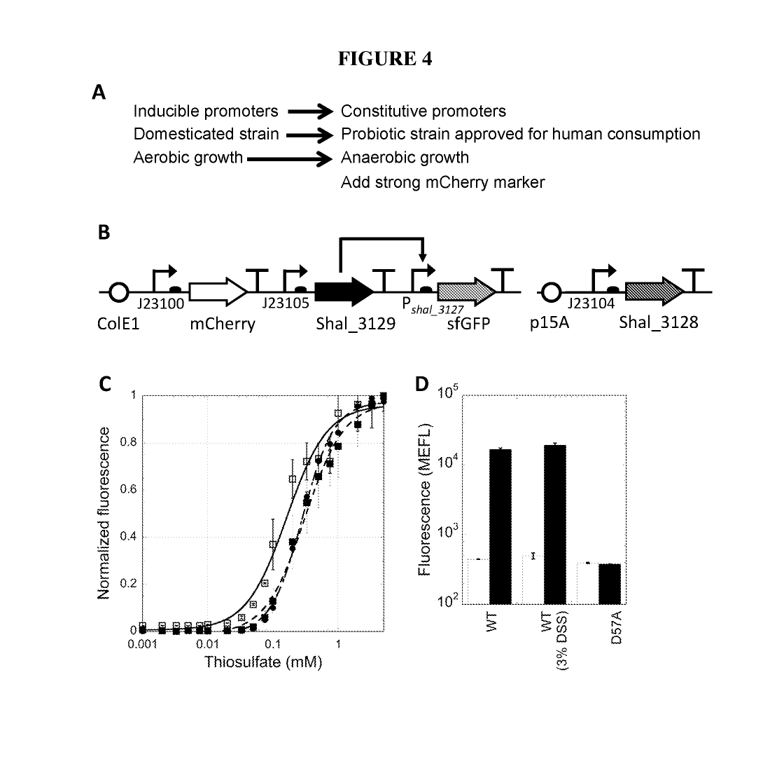

FIG. 4 shows optimization efforts for the thiosulfate biosensor for use in vivo, wherein the host was replaced with a probiotic strain of E. coli, the inducible promoters were replaced with strong constitutive promoters, and the promoter strength of the constitutive mCherry reporter was increased to facilitate identification of sensor bacteria from the complex microbial community of the mammalian gut. The optimized sensor has similar sensitivity to thiosulfate as the inducible system in BW28357 and does not appear to be sensitive to the presence of oxygen or DSS (FIGS. 4C and D).

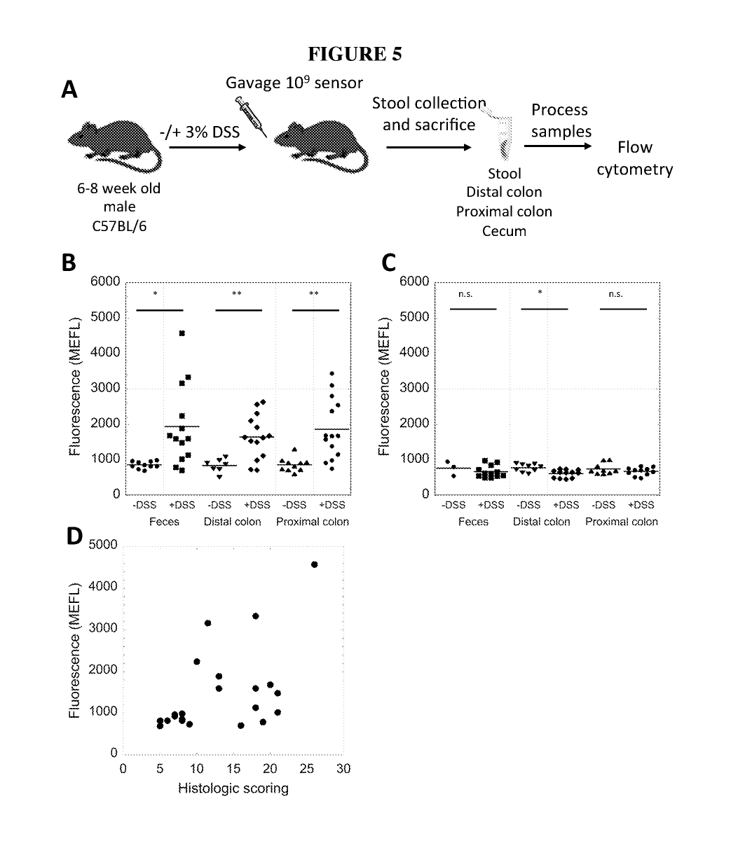

FIG. 5 shows proof of concept work in vivo wherein mice were gavaged with the biosensor bacteria and after 6 hours, gut samples were collected from the mice, processed, and analyzed by flow cytometry to measure GFP production. These results show that histology score and thiosulfate sensor output are correlated, indicating that thiosulfate is indeed a biomarker of inflammation and that the sensor can detect physiologically relevant thiosulfate concentrations in vivo.

The following are incorporated by reference herein in its entirety for all purposes:
Snijder, P. M., Frenay, A. R., de Boer, R. A., Pasch, A., Hillebrands, J. L., Leuvenink, H. G. D. and van Goor, H. Exogenous administration of thiosulfate, a donor of hydrogen sulfide, attenuates angiotensin II-induced hypertensive heart disease in rats. British Journal of Pharmacology. 2015; 172:1494-1504.
Tokuda K, Kida K, Marutani E, et al. Inhaled Hydrogen Sulfide Prevents Endotoxin-Induced Systemic Inflammation and Improves Survival by Altering Sulfide Metabolism in Mice. Antioxidants & Redox Signaling. 2012; 17(1):11-21.
Fredrickson, J. K, Romine, M. F., Beliaev, A. S., et al. Towards environmental systems biology of Shewanella. Nat Rev Microbiol. 2008; 6(8):592-603.
62/157,293, IDENTIFYING LIGANDS FROM BACTERIAL SENSORS, May 5, 2015

1) A genetically engineered bacteria, said bacteria expressing:
a) a two component sensor system (TCS) comprising:
i) a thiosulfate-sensing sensor kinase (SK) gene comprising a ligand binding domain operably coupled to a kinase domain; and,
ii) a cognate response regulator (RR) gene comprising a receiver domain operably coupled to an DNA binding domain (DBD); and,
b) an output promoter comprising a DNA binding site that binds said DBD and is operably coupled to a reporter gene.

2) The bacteria of claim 1, wherein said SK gene or said RR gene or both genes are encoded on an expression vector, an inducible expression vector, and/or a constitutive expression vector.

3) (canceled)

4) (canceled)

5) The bacteria of claim 1, wherein said SK gene or said RR gene or both genes integrated into a chromosome of said bacteria.

6) The bacteria of claim 1, wherein said SK gene and said RR gene are encoded in a single operon.

7) The bacteria of claim 1, wherein said output promoter and said reporter gene are encoded on a plasmid.

8) The bacteria of claim 1, wherein said output promoter and said reporter gene are integrated into a chromosome of said bacteria.

9) The bacteria of claim 1, wherein said SK gene and said RR gene are from Shewanella halifaxensis.

10) The bacteria of claim 1, comprising SEQ ID NO. 1 and SEQ ID NO. 2.

11) (canceled)

12) The bacteria of claim 1, wherein said reporter gene encodes a fluorescent protein.

13) The bacteria of claim 1, wherein said reporter gene encodes green fluorescent protein, red fluorescent protein, far red fluorescent protein, blue fluorescent protein, orange fluorescent protein, yellow fluorescent protein, mCHERRY, mORANGE, mCITRINE, VENUS, YPET, EMERALD, or CERULEAN.

14) The bacteria of claim 1, wherein said bacteria is probiotic for use in humans.

15) A method of detecting thiosulfate, comprising:
i) combining a test sample with the bacteria of claim 1; and,
ii) measuring expression of said reporter gene, wherein a change in a level of expression of said reporter gene as compared to a control sample lacking thiosulfate indicates that said test sample contains thiosulfate.

16) A method of detecting excess thiosulfate in a patient, comprising i) administering the bacteria of claim 1 to a patient, ii) collecting a gut or stool sample from said patient; and, iii) measuring expression of said reporter gene in said gut or stool sample, wherein a change in level of expression of said reporter gene over a normal level in a normal patient indicates that said patient has excess thiosulfate.

17) A method of measuring thiosulfate levels in a patient, comprising:
a) combining a gut or stool sample with a thiosulfate reporter bacteria comprising:
i) a thiosulfate-sensing sensor kinase (SK) gene encoding an SK protein comprising a ligand binding domain that binds thiosulfate and activates a kinase domain,
ii) a cognate RR gene encoding an RR protein comprising a receiver domain operably coupled to an DNA binding domain (DBD), wherein said cognate RR protein is activated by said activated kinase domain phosphorylating said receiver domain and
iii) a DNA binding site that binds said DBD of said cognate activated RR protein, wherein said DNA binding site is operably coupled to a reporter gene;
b) measuring expression of said reporter gene; and,
c) correlating a measured level of reporter gene expression with a level of thiosulfate using a standard curve.

18) A method of screening for gut inflammation, comprising i) administering the bacteria of claim 1 to a patient, ii) collecting a stool sample from said patient, and iii) measuring activity of said reporter gene in said stool sample, wherein a change in reporter gene expression over a normal level in a normal patient indicates that said patient has gut inflammation.

19) (canceled)

20) (canceled)

21) A treatment method, comprising administering a bacteria to a patient having excess thiosulfate, wherein said bacteria overexpresses a heterologous thiosulfate sensor system comprising:
i) a thiosulfate-sensing sensor kinase (SK) comprising a ligand binding domain operably coupled to a kinase domain; and,
ii) a cognate response regulator (RR) comprising a receiver domain operably coupled to an DNA binding domain (DBD);
a DNA binding site that binds said DBD that is operably coupled to either a reporter gene or a therapeutic protein gene, and
wherein said DBD is operably coupled to a therapeutic protein.

22) A treatment method of claim 20, said therapeutic protein that reduces inflammation.
